(19) United States
(12) Patent Application Publication
(10) Pub. No.: US 2019/0038463 A1
(43) Pub. Date: Feb. 7, 2019

(54) OPHTHALAMIC IMPLANT FOR REDUCTION OF INTRAOCULAR PRESSURE IN GLAUCOMATOUS EYES AND METHOD OF USE

(71) Applicant: AQ BIOMED, LLC, Boulder, CO (US)

(72) Inventor: Jim KASIC, Boulder, CO (US)

(73) Assignee: AQ BIOMED, LLC, Boulder, CO (US)

(21) Appl. No.: 16/073,713

(22) PCT Filed: Jan. 30, 2017
(86) PCT No.: PCT/US2017/015561

§ 371 Date: Jul. 27, 2018

Related U.S. Application Data
(60) Provisional application No. 62/288,232, filed on Jan. 28, 2016.

(51) Int. Cl. A61F 9/007; A61F 9/00

(52) U.S. Cl. CPC A61F 9/00781; A61F 2/16; A61F 2210/0014; A61F 9/0017

(57) ABSTRACT

The disclosure provides an ophthalmic implant for reducing intraocular pressure in an eye tissue. The implant may include a proximal portion including a fluid channel. The implant may also include a distal portion including a filtration capsule forming member. The filtration capsule forming member may include at least one flat surface, and configured to form a cylindrical filtration capsule from the eye tissue surrounding the filtration capsule forming member when implanted. The fluid channel is configured to be fluidly connected to the cylindrical filtration capsule.

### CROSS-REFERENCE TO RELATED PATENT APPLICATION

This patent application claims the benefit of U.S. Patent Application No. 62/288,232, entitled “OPHTHALMIC IMPLANT FOR REDUCTION OF INTRAOCULAR PRESSURE IN GLAUCOMATOUS EYES AND METHOD OF USE,” filed on Jan. 28, 2016 under 35 U.S.C. § 119(e), which is incorporated herein by reference in its entirety.

### FIELD

The present disclosure is directed to an ophthalmic implant for reduction of intraocular pressure in glaucomatous eyes and method of use.

### BACKGROUND

Glaucoma is a disease characterized by high pressure inside the eye, leading to the loss of retinal nerve fibers and a corresponding loss of vision. Glaucoma, therefore, is a disease affecting the optic nerve, the nerve bundle which carries visual information to the brain.

The eyeball is basically a rigid sphere filled with fluid. Positive pressure inside the eye is needed to keep the retina attached to the back of the eye. Pressure is maintained by fluid production from a bilayer of cells on top of the ciliary body, which is located adjacent to the iris root in the eye. This clear fluid, called aqueous humor, carries nutrients to the lens and cornea of the eye, both of which have no blood supply.

The shape of the front part of the eye is maintained by aqueous humor production. The ciliary body is located behind the colored part of the eye (iris). Fibrous strands called zonule fibers are attached to the ciliary body support the lens. Tension from the rigid structure of the eye, transferred to these zonules, deforms the lens and focuses the image onto the retina. Radial muscles behind the ciliary body contract and release tension on the zonules, allowing the lens to round up and focus near images onto the retina. Aqueous humor flows into the front of the eye through the pupil and drains out of the eye through the trabecular meshwork. The trabecular meshwork is a spongy mass of tiny channels located in the drainage angle, between the clear covering of the eye (cornea) and the colored part (iris) at the location where the iris meets the white outer covering of the eye (sclera). The fluid drains from the meshwork into a small canal, called Schlemm's canal, which is connected to the bloodstream at the venous return from the eye.

Glaucoma is the leading cause of blindness in third world countries, and the leading cause of preventable blindness in industrial countries. It affects approximately 2% of the entire population; blacks and native Americans are disproportionately affected, with higher occurrence of the disease than others. Early signs of the disease are often observed as enlargement and cupping of the physiological blind spot which is the point where the optic nerve leaves the eyeball and projects to the brain. Blind spots in the superior and inferior visual fields (called arcuate scotomas) correspond to the loss of nerve cells. In later stages, more visual field losses eventually result in total blindness. If a drainage angle becomes blocked, fluid pressure transferred throughout the eye increases, damaging the optic nerve—the part of the eye responsible for transforming images into nerve impulses the brain can translate. This damage results in partial or complete blindness.

In acute angle-closure glaucoma, eye pressure builds up rapidly. This type of glaucoma commonly occurs in individuals who have narrow anterior chamber angles. In these cases, aqueous fluid behind the iris cannot pass through the pupil and therefore pushes the iris forward, preventing aqueous drainage through the angle. In cases of acute angle closure glaucoma, one may experience blurred vision, halos around lights, pain, nausea, and vomiting. If pressure within the eye is not immediately relieved, blindness may result in a matter of days. Migration of pigmented epithelial cells in the eye, either congenital or following blunt trauma, can occlude angle structures, quickly elevating pressure in the eye. Primary open-angle glaucoma has a longer time course and many components that exacerbate the condition. The end effect is the same. Secondary glaucoma can occur from inflammation, degeneration, trauma, or tumor growth within the eye. Left uncontrolled, high intraocular pressure leads to blindness.

Surgical treatment of glaucoma using a shunt implant to vent aqueous humor from the anterior chamber to a subconjunctival bleb is warranted in some cases. To date, currently available shunt implants consist of a fluid channel or conduit attached to the surface of a large plate. The conduit carries fluid from the anterior chamber to the surface of the plate. A cellular capsule forms around that plate and once inflated with aqueous humor from the anterior chamber forms a large blister or bleb around the plate on the outer surface of the eye. The geometry of the blister's surface takes on the shape of the plate. The cellular capsule forming the bleb wall filters fluid to the subconjunctival space and provides resistance to fluid flow so pressure in the eye will not be too low.

Limitations of conventional implantable devices have been described in, for example, U.S. Pat. Nos. 6,962,573 and 7,806,847, each of which is incorporated herein by reference in its entirety. A large volume blister causes most of the post-surgical complications. Accordingly, there remains a need in developing new implant devices, including additional implant geometries, surgical methods with reduced invasiveness (e.g. minimally invasive), methods of controlling diffusion and/or the rate thereof, and introducing clotting agents. These and other needs are provided in this disclosure.

### BRIEF SUMMARY

The disclosure provides an implant for reducing intraocular pressure and a device for loading the implant with minimum invasiveness into the eye.

In an embodiment, an ophthalmic implant is provided for reducing intraocular pressure in an eye tissue. The implant may include a proximal portion including a fluid channel. The implant may also include a distal portion including a filtration capsule forming member. The filtration capsule forming member may include at least one flat surface, and configured to form a cylindrical filtration capsule from the eye tissue surrounding the filtration capsule forming member when implanted. The fluid channel is configured to be fluidly connected to the cylindrical filtration capsule.

In some embodiments, the filtration capsule forming member includes one of a plurality of lateral members, a boundary member, and a plurality of bridging members.

In some embodiments, each of a plurality of lateral members, a boundary member, and a plurality of bridging members includes a cross-section having a square or rectangular shape.

In some embodiments, the distal portion includes a plurality of lateral members within a circular or an elliptical contour.

In some embodiments, the filtration capsule forming member includes the plurality of lateral members.

In some embodiments, the distal portion includes a boundary member, a longitudinal bridging member, and a plurality of transverse bridging members connected between the boundary member and the longitudinal bridging member.

In some embodiments, the filtration capsule forming member includes the boundary member, the longitudinal bridging member, and the plurality of transverse bridging members.

In another embodiment, an implantation device is provided for implanting an ophthalmic implant into an eye tissue. The device may include an elongated member including a distal portion and a proximal portion, and a lumen between the distal portion and the proximal portion.

In some embodiments, the ophthalmic implant is placed in the lumen. The distal portion of the ophthalmic implant is in the distal portion of the needle, and the proximal portion of the ophthalmic implant is in the proximal portion of the needle.

In some embodiments, the elongated member includes a needle.

In some embodiments, the device further includes a loading component configured to mechanically push the ophthalmic implant into the lumen.

In some embodiments, the loading component is configured to guide the ophthalmic implant toward the distal portion of the elongated member.

In some embodiments, the loading component comprises a metal wire configured to pass through the fluid channel of the ophthalmic implant.

In some embodiments, the ophthalmic implant comprises a stop end configured for the wire to be pushed against to allow the ophthalmic implant to be pushed into the lumen.

In some embodiments, the loading component comprises a metal wire configured to stretch the ophthalmic implant to shape the ophthalmic implant into a rod-like assembly including the implant and wire, such that the rod-like assembly can be pushed into the lumen of the elongated member. The loading component can be made of any other material that can be used to push the rod-like assembly into the lumen of the elongated member.

In some embodiments, the ophthalmic implant comprises a shape memory material. In some instances, the shape memory material can be a shape memory polymer.

In some embodiments, a method is provided for implanting the ophthalmic implant. The method may include inserting an elongated member loaded with the ophthalmic implant of claim 1 into an eye ball. The ophthalmic implant is deformed from an original shape to fit inside the elongated member. The method may also include withdrawing the elongated member from the eye ball to leave the ophthalmic implant in the eye ball. The ophthalmic implant returns to the original shape.

In some embodiments, the method may further include loading the ophthalmic implant into a lumen of an elongated member by pushing a loading component. The ophthalmic implant is deformed from the original shape to fit inside the lumen.

In some embodiments, the method may further include pulling out the loading component after withdrawing the elongated member.

In a further embodiment, an ophthalmic implant is provided for reducing intraocular pressure in an eye tissue. The implant may include a proximal portion including a fluid channel. The implant may also include a distal portion including a filtration capsule forming member. The filtration capsule forming member configured to form a cylindrical filtration capsule when the ophthalmic implant implanted. The distal portion may include a shape memory material. The fluid channel is configured to be fluidly connected to the cylindrical filtration capsule when formed from the eye tissue surrounding the filtration capsule forming member.

In some embodiments, the filtration capsule forming member has at least on one flat surface.

Additional embodiments and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure, wherein:

FIG. 1A is a perspective view of an ophthalmic implant including a flat structure coupled to a fluid channel in a first embodiment of the disclosure.

FIG. 1B is a cross-sectional view illustrating a filtration capsule formed from the tissue surrounding a segment of the lateral member of FIG. 1A in accordance with embodiments of the disclosure.

FIG. 2 is a top view of an ophthalmic implant including a flat structure coupled to a fluid channel in a second embodiment of the disclosure.

FIG. 3 is a top view of an ophthalmic implant including a flat structure coupled to a fluid channel in a third embodiment of the disclosure.

FIG. 4 is a top view of an ophthalmic implant including a flat structure coupled to a fluid channel in a fourth embodiment of the disclosure.

FIG. 5 is a top view of an ophthalmic implant including a flat structure coupled to a fluid channel in a fifth embodiment of the disclosure.

FIG. 6 is a side view of an ophthalmic implant including a flat structure coupled to a fluid channel being stretched by wires in accordance with embodiments of the disclosure.

FIG. 7 is a side view of the ophthalmic implant inserted of FIG. 6 in a needle in accordance with embodiments of the disclosure.

FIG. 8 illustrates that the needle including the ophthalmic implant of FIG. 7 inserted into an eye in accordance with embodiments of the disclosure.

FIG. 9 illustrates that the needle of FIG. 7 withdrawn from the eye in accordance with embodiments of the disclosure.

FIG. 10 is a top view of the ophthalmic implant in a sixth embodiment of the disclosure.

FIG. 11 is a perspective view of the ophthalmic implant in a seventh embodiment of the disclosure.

### DETAILED DESCRIPTION

The disclosure may be understood by reference to the following detailed description, taken in conjunction with the drawings as described below. It is noted that, for purposes of illustrative clarity, certain elements in various drawings may not be drawn to scale.

The disclosure provides an ophthalmic implant for implantation into an eye tissue or an eyeball. When implanted, the ophthalmic implant causes an increase in the flow of aqueous humor from the eyeball into a subconjunctival space, and reduces intraocular pressure.

The ophthalmic implant includes a proximal portion including a fluid channel (e.g., a tube) coupled to a distal portion including a two-dimensional structure. In various aspects, the two-dimensional structure can be a flat structure, which is also referred to a filtration capsule forming member. In some embodiments, the flat structure includes a number of lateral members. In some embodiments, the flat structure includes a number of bridging members and a boundary member. When the ophthalmic implant is implanted in the eyeball, at least a portion of the proximal portion is implanted in the eyeball. The fluid channel is configured to pass through the eyeball into the subconjunctival space. The distal portion resides in the subconjunctival space.

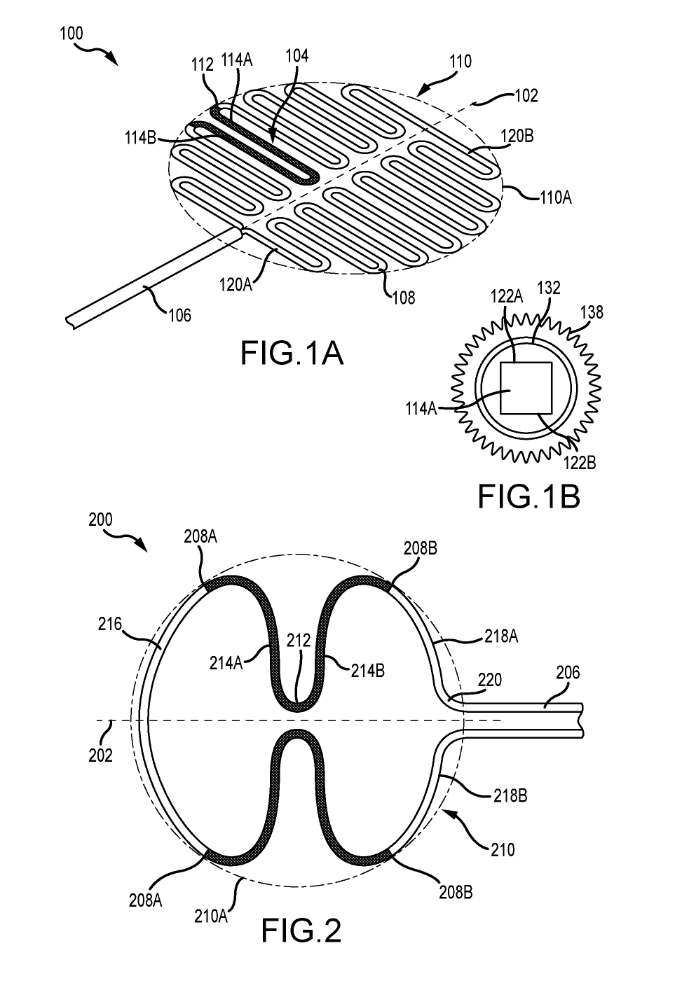

FIG. 1A is a perspective view of an illustrative embodiment of the ophthalmic implant. The ophthalmic implant 100 has a proximal portion 100A including a fluid channel 106, and a distal portion 100B including a filtration capsule forming member or a flat structure 110. In the non-limiting example of FIG. 1A, the fluid channel 106 extends along a center line 102.

The flat structure 110 is a two-dimensional structure including at least one flat surface 122A or 122B. The flat structure 110 can include two flat surfaces 122A-B on opposing sides of the flat structure 110. The flat structure 110 includes a plurality of lateral members 104 arranged in a two-dimensional configuration.

In various aspects, the lateral members 104 of the ophthalmic implant 100 can include mostly straight sections. The flat structure 110 is a single integral component including a plurality of lateral members 104. In FIG. 1A, the flat structure 110 includes twelve lateral members 104. One lateral member is connected to a neighboring lateral member. Six lateral members 104 are arranged on one side of the center line 102 and another six lateral members 104 are arranged on an opposite side of the center line 102. Though the lateral members 104 are arranged substantially symmetrically with respect to the central line 102, it will be recognized that the lateral members 104 may not be arranged symmetrically.

The flat structure 110 also includes a proximal end section 120A coupled to the fluid channel 106 and a distal end section 120B opposite to the fluid channel 106. The distal end portion 120B is continuously connected between two opposite lateral members. The proximal end section 120A and the distal end section 120B are non-curved in this embodiment.

The lateral members 104 can be spaced apart from each other. Each lateral member 104 can be continuously connected to the neighboring lateral members 104 at its outer ends 108. The lateral members 104 may have varying length, such that the outer ends 108 of the lateral members 108 may be spaced apart along or near a contour 110A.

Each lateral member 104 includes first and second segments 114A and 114B that are continuously connected at a single inner end 112 of the lateral member 104 and are separated at the outer ends 108A-B of each lateral member 104. For example, the outer end 108A of the first segment of a first lateral member is continuously connected to the outer end 108B of the segment 114A of the neighboring second lateral member 104, while the outer end 108B of the second segment 114B of the first lateral member is continuously connected to the outer end 108A of the segment 114A of the neighboring third lateral member 104. Each segment of the lateral member 104 extends outwardly away from the single inner end 112 near the center line 102.

Each segment 114A or 114B of lateral member 104 may have a cross-section that may be in a rectangular shape or a square shape, or any other shapes such that the lateral member has at least one flat surface. In some embodiments, the lateral members 104 may have two opposite flat surfaces 122A and 122B substantially parallel to each other. The flat structure 110 with at least one flat surface 122A or 122B is easy to fabricate.

In some embodiments, the segment 114A or 114B of the lateral member 104 may have a thickness that is less than the inner diameter of the fluid channel 106.

When ophthalmic implant 100 is implanted, a filtration capsule 132 forms from the eye tissue 138 surrounding the surfaces 122A-B of the segment 114A of the distal portion 100B of the ophthalmic implant 100, as shown in FIG. 1B. The filtration capsule 132 can take a period of time to form along the distal portion 100B of ophthalmic implant 100 (e.g. a period of days, a week, or a period of weeks). When liquid, such as aqueous humor, flows through the fluid channel 106 into the distal portion 100B of the ophthalmic implant 100, the liquid enters the space 134 between the proximal portion 100A of the ophthalmic implant 100 and the tissue to inflate the filtration capsule 132.

In various embodiments, a first portion of the filtration capsule can be formed around the lateral members 104 of the filtration capsule forming member. A second portion of the filtration capsule can also be formed around the distal end section 120B of the filtration capsule forming member. A third portion of the filtration capsule can also be formed around the proximal end section 120A of the filtration capsule forming member. In this embodiment, the filtration capsule including the first, second and third portions may be substantially cylindrical. In some embodiments, the filtration capsule is substantially cylindrical. The cylindrical filtration capsule can exist around all, or a portion of, the distal portion 100B of the ophthalmic implant 100.

The aqueous humor can flow between the lateral members and the eye tissue to inflate the filtration capsule. The filtration capsule is often referred to as a bleb or a blist. The filtration capsule allows the aqueous humor to pass into the subconjunctival space. The filtration capsule acts like a membrane that filters the aqueous humor into the subconjunctival space. The filtration capsule also provides resistance to the flow of aqueous humor so that pressure in the eye will not be too low.

The disclosed implant structures or devices for reducing intraocular pressure provide a more effective and customizable, yet economical solution for reduction of intraocular pressure. The disclosed implant structures can help reduce collagen deposition. For the cylindrical filtration capsule formed over the distal portion including filtration capsule forming members, such as lateral members, the surface tension is reduced compared to the spherical cellular membrane, such that collagen deposition is reduced in the cylindrical filtration capsule.

In contrast, current large-plate shunt devices forming a hemisphere blister or geometry do not reduce collagen deposition and affect fluid filtering. The hemisphere blister minimizes surface area while maximizing volume. This is a disfavored design because the goal is increasing the surface area so that aqueous or liquid can be vented to the orbit and control pressure inside the eye. Surface tension is proportional to the radius of a spherical blister according to Laplace's law. The larger the surface tension is applied to the cellular capsule or filtration capsule, the more the membrane will stretch until the cohesive force of the capsule cannot match the applied force. The hemisphere capsule thereby ruptures like a balloon. The body's reaction is to reinforce the spherical capsule with collagen deposition, which would make the hemisphere capsule thicker and less effective at filtering fluid. In particular, all large plate devices with the spherical blister geometry require a maturation period during which the capsule thickens, which makes the capsule less effective at filtering or venting fluid and sometimes fails early due to the build-up of fibrous tissue in the capsule. The cylindrical filtration capsule solves this problem by forming thinner capsules than the spherical capsules without reduced collagen deposition.

The pressure in the ophthalmic implant may be controlled in various ways. In an embodiment, the pressure reduction may vary with the width of the lateral members of the distal portion or the number of lateral members. A portion of the cylindrical filtration capsule can be formed from the tissue surrounding each lateral member. For example, an increased width of the lateral members may increase the surface area and thus the resistance to the flow, and thus provide resistance to pressure reduction. Also, an increase in the number of lateral members may increase the surface area and thus increase resistance to the flow, and thus provides resistance to pressure reduction.

The ophthalmic implant may have various alternative embodiments. As shown in FIG. 2, an ophthalmic implant 200 includes a distal portion including a filtration capsule forming member or a flat structure 210 coupled to a fluid channel 206. The flat structure 210 may include two curved lateral members 204, which are shaped differently from the substantially non-curved or linear shaped lateral members 104 in the flat structure 110.

The flat structure 210 is in a generally circular or elliptical contour 210A having a diameter D as marked. The lateral members have varying lengths such that the outer ends 208A-B of the lateral members 204 are spaced apart along the contour 210A of the flat structure 210.

The lateral members 204 are arranged on opposite sides of a center line 202 and are substantially symmetric to the central line 202 which splits the flat structure 200 into half. Though the lateral members 204 are arranged substantially symmetrically with respect to the central line 202, it will be recognized that the lateral members 204 may not be arranged symmetrically.

Each lateral member 204 includes two segments 214A and 214B that are continuously connected at a single inner end 212 of each lateral member 204 and are separated at two outer ends 208A-B of each lateral member 204.

The flat structure 210 is a single integral component including two lateral members 204 and curved distal sections 216 and curved proximal 218A-B in this embodiment. Opposite to the fluid channel 206 with respect to the lateral members 204, curved distal section 216 includes two ends 222A-B, which are continuously connected to the segments 214A and 214D of each of the lateral members 204 near the outer ends 208. Near the fluid channel 206, two curved proximal sections 218A-B are connected to the end 220 of the fluid channel 306. Each of the curved proximal sections 218A-B is continuously connected to the respective segment 214B or 214C of the lateral members 204 near the respective outer end 208B.

The curved proximal sections 218A-B and curved distal section 216 may be near or along the circular or elliptical contour 210A of the flat structure 210. It will be appreciated by those skilled in the art that the shape of the contour of the flat structure may vary.

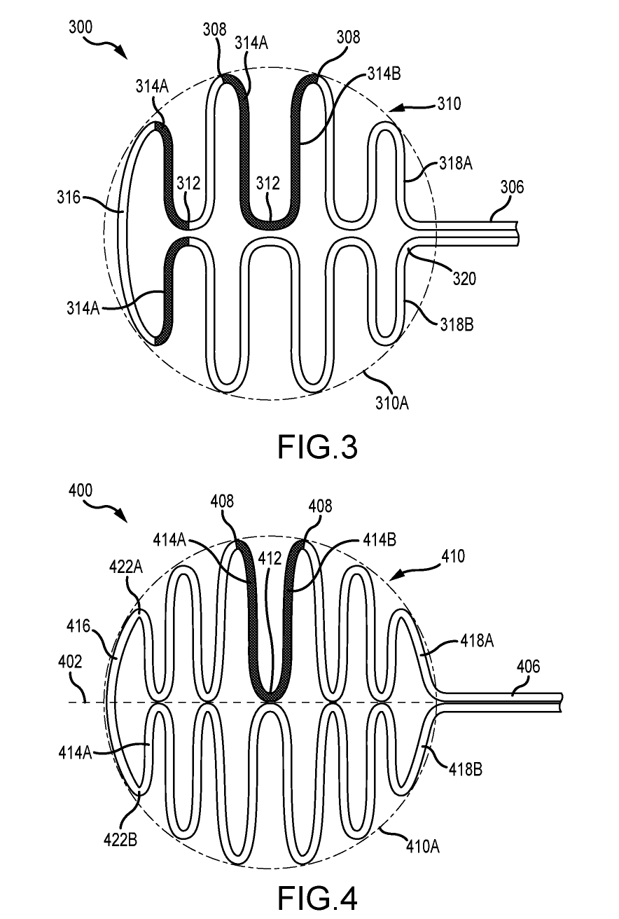

The number of lateral members may vary in the flat structure. As shown in FIG. 3, an ophthalmic implant 300 includes a distal portion including a filtration capsule forming member or a flat structure 310 coupled to a fluid channel 306. The flat structure 310 may include three lateral members 304 on each side of a center line 302. Though the lateral members 304 are arranged substantially symmetrically with respect to the central line 302, it will be recognized that the lateral members 304 may not be arranged symmetrically.

The flat structure 310 is a single integral component including six lateral members 304 and curved distal section 316 and curved proximal sections 318A-B in this embodiment. Similar to the embodiment shown in FIG. 2, opposite to the fluid channel 306 with respect to the lateral members 304, curved distal section 316 includes two ends 322A-B, and each end of the curved section 316 is continuously connected to the segment 314A of the most left lateral members 304 by outer ends 308. By the fluid channel 306, two curved proximal sections 318A-B are connected to the fluid channel 306. Each of the curved proximal sections 318A-B is continuously connected to the segment 314A of the most right lateral member 304 near the outer end 308.

The curved proximal sections 318A-B and curved distal section 316 may be near or along the circular or elliptical contour of the flat structure 310. Also, the outer ends 308 of the lateral members 304 may be spaced apart along or near the circular or elliptical contour 310A of the flat structure 310. It will be appreciated by those skilled in the art that the shape of the contour of the flat structure may vary.

As shown in FIG. 4, an ophthalmic implant 400 includes a distal portion including a filtration capsule forming member or a flat structure 410 coupled to a fluid channel 406. The flat structure 410 may include five lateral members 404 on each side of a center line 402. Five lateral members 404 are arranged on one side of the center line 402 and another five lateral members 104 are arranged on an opposite side of the center line 402. Though the lateral members 404 are arranged substantially symmetrically with respect to the central line 402, it will be recognized that the lateral members 404 may not be arranged symmetrically.

Similar to the embodiments shown in FIGS. 2-3, the flat structure 410 is a single integral component including ten lateral members and curved sections 416 and 418A-B. opposite to the fluid channel 406 with respect to the lateral members 404, curved distal section 416 includes two ends 422A-B, and each end 422A or 422B of the curved distal section 416 is continuously connected to the segment 414A of the most left lateral members 404 by outer ends 408. By the fluid channel 406, two curved proximal sections 418A-B are connected to the fluid channel 406. Each of the curved proximal sections 418A-B is continuously connected to the segment 414A of the most right lateral member 404 near the outer end 408.

The curved proximal sections 418A-B and distal section 416 as well as the outer ends 408 may be near or along the circular or elliptical contour 410A of the flat structure 410, similar to that shown in FIGS. 1-3. It will be appreciated by those skilled in the art that the shape of the contour of the flat structure may vary.

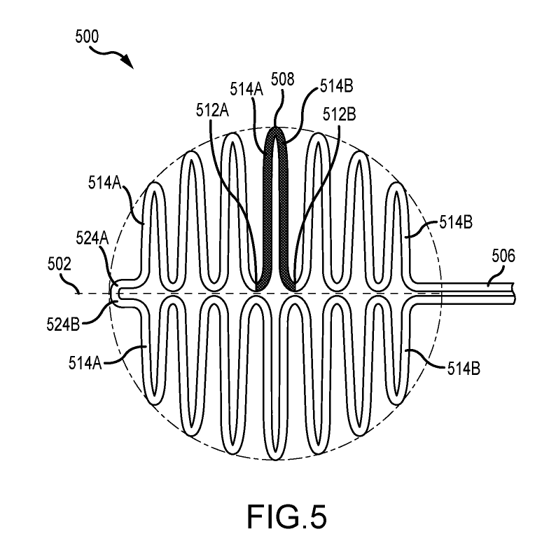

As shown in FIG. 5, the flat structure may have an alternative end configuration from FIGS. 2-4. In this embodiment, an ophthalmic implant 500 includes a distal portion including a filtration capsule forming member or a flat structure 510 coupled to a fluid channel 506. The flat structure 510 may include fourteen lateral members 504. Seven lateral members 504 are arranged on one side of a center line 502 and another seven lateral members 504 are arranged on an opposite side of the center line 502. Though the lateral members 504 are arranged substantially symmetrically with respect to the central line 502, it will be recognized that the lateral members 504 may not be arranged symmetrically.

Each lateral member 504 includes two segments 514A and 514B connected near an outer end 508 of the lateral member 504 and the two segments 514A and 514B are separated at inner ends 512A-B near a centerline 502. The inner end 512A of one lateral member 504 is continuously connected to the inner end 512B of the neighboring lateral member 504. Opposite to the fluid channel 506, the ends 524A-B of the segments 514A of two opposed lateral members 504 are connected along the center line 506. By the fluid channel 506, the segments 514B of the two opposed lateral members 504 are connected to the fluid channel 506.

Outer ends 520 of the lateral members 504 may be spaced along or near the contour 510A of the flat structure 510. The contour of the flat structure 510 may be circular or other shape, such as elliptical. It will be appreciated by those skilled in the art that the shape of the contour of the flat structure may vary.

In some embodiments, the segments 114A-B in the lateral members 104 may be substantially parallel to each other, as shown in FIG. 1A. In alternative embodiments, the segments, e.g. 214A-B, 314A-B, or 414A-F, may be curved and continuously connected at a single inner end. In alternative embodiments, the segments, e.g. 514A-B, may be curved and continuously connected at a single outer end.

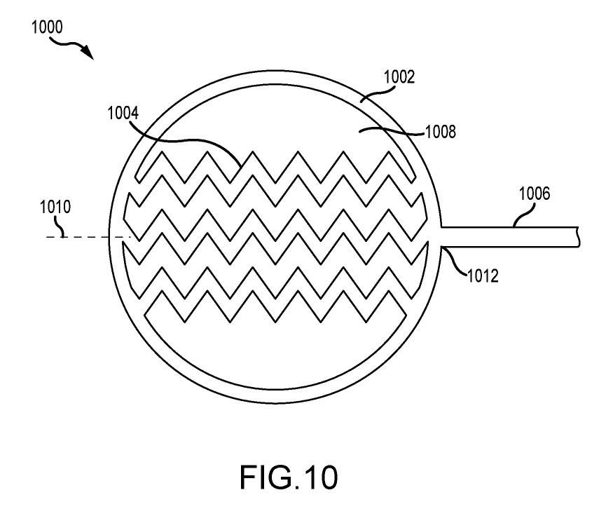

FIG. 10 is a top view of an ophthalmic implant in a sixth embodiment. As shown, an implant 1000 includes a distal portion 1002, including a filtration capsule forming member or a two-dimensional flat structure, coupled to a proximal portion 1006 (e.g. a fluid channel). The fluid channel 1006 extends along a longitudinal axis 1010. The distal portion or flat structure 1002 includes a boundary member 1012 and a number of bridging members 1004, which connect between two spaced-apart points of the boundary member 1012. In this embodiment, there are three bridging members. It will be appreciated by those skilled in the art that the number of bridging members may vary.

The boundary member 1012 may be a closed loop in a circular shape in this embodiment. It will be appreciated by those skilled in the art that the shape of the boundary member 1012 may vary. The boundary member may also be an open loop.

The bridging members 1004 may be shaped like a saw tooth which helps increase the surface area. The bridging members 1004 can also be extended when the boundary member 1012 is stretched along the longitudinal axis 1110. The bridging members 1004 are separated from each other by a space 1108 to allow forming of a portion of the cylindrical filtration capsule around the bridging members 1004 or the boundary member 1012.

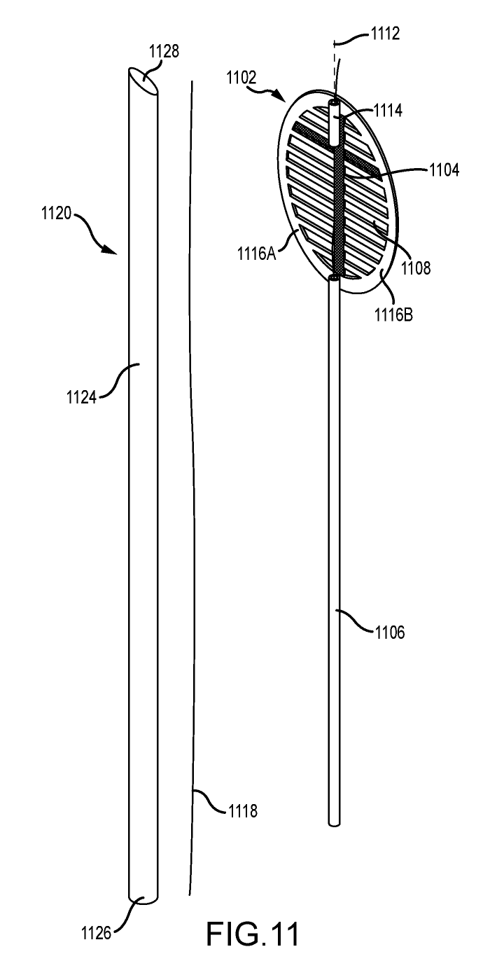

FIG. 11 is a perspective view of the ophthalmic implant in a seventh embodiment of the disclosure. As shown in FIG. 11, an implant 1100 includes a proximal portion 1106 (e.g. fluid channel) coupled to a distal portion 1102 including a filtration capsule forming member or a flat structure. The fluid channel 1106 extends along a longitudinal axis 1112.

The flat structure 1102 includes a boundary member 1116 including two curved sections 1116A and 1116B connected to each other. The boundary member 1116 may be a closed loop in a circular shape in this embodiment. It will be appreciated by those skilled in the art that the shape of the boundary member 1116 may vary. In some embodiments, the boundary member 1116 may be an open loop.

The flat structure 1102 also includes a longitudinal bridging member (e.g. rail) 1122, which is connected between opposite points of the boundary member 1116 along the longitudinal axis 1112. The longitudinal bridging member 1122 is in a straight bar shape having a rectangular or square cross-section. It will be appreciated by those skilled in the art that the shape of the longitudinal bridging member 1122 may vary.

The flat structure 1102 further includes a number of transverse bridging members (e.g. rails) 1104 along a transverse axis to the longitudinal axis 1112. Each transverse bridging member 1104 is connected between the boundary member 1116 and the longitudinal bridging member 1122. In some embodiments, the transverse bridging members 1104 are connected between the curved outer sections 1116A and 1116B and the longitudinal bridging member 1112. The transverse bridging member 1104 is in a straight bar shape having a rectangular or square cross-section. It will be appreciated by those skilled in the art that the shape of the transverse bridging member 1104 may vary.

The implant 1100 also includes a stop end 1114 configured for holding an end of a loading component, such as a wire 1118, such that the wire 1118 can push the implant 1100 into a lumen 1124 of a needle 1120. The stop end 1114 is located near the top end opposite to the fluid channel 1106.

Various embodiments of the distal portion or the flat structure, such as shown in FIGS. 1-5 and 10-11, are provided to increase the surface area. The filtration capsule can be formed from the eye tissue surrounding the flat structure or the filtration capsule forming member. A portion of the filtration capsule can be formed around each lateral member, as shown in FIGS. 1-5, or each transverse bridging member, or each boundary member, as shown in FIGS. 10-11. The bridging member may be in a straight bar shape, as shown in FIG. 11, or in a saw-tooth shape as shown in FIG. 10.

In various embodiments, the flat structure, such as shown in FIGS. 1-5 and 10-11 can provide improved ease of manufacturing.

Variations

It will be appreciated by those skilled in the art that the flat structure may vary in size or shape. The number of lateral members or bridging members may vary. The dimensions of the lateral members or bridging members, such as width, thickness, segment length, spacing between the lateral members or bridging members, may also vary. The lateral members or bridging members may also vary in cross-sectional shape, configuration of segments, and/or connections among to other lateral members.

The dimensions of the implant, such as the dimensions of the lateral members and contour of the flat structure, or the dimensions of the bridging members and boundary members, can affect the permeability of the filtration capsule, which affects the intraocular pressure.

In some embodiments, the thickness of the filtration capsule may increase with the dimensions of the implant. Pressured flow measurements of isolated blebs show that aqueous outflow per square millimeter of capsule is proportional to the thickness of the filtration capsule. The capsule thickness can be measured by scanning electron microscope (SEM) for providing the relationship between the capsule thickness and the implant diameter.

The total resistance to flow for the filtration capsule is proportional to the thickness of the filtration capsule. Thinner filtration capsules with the same surface area can filter more aqueous humor than thicker filtration capsules around large implants. This implant allows reaching a low intraocular pressure range after eye surgery without inducing hypotony and maintaining the pressure for a long period of time.

The thickness of the filtration capsule may be selected for reducing the intraocular pressure while preventing the pressure to become too low. In order to control the thickness of the filtration capsule, the dimensions of the implant may be selected in appropriate ranges.

In some embodiments, the contour of the flat structure or the boundary member may have a diameter less than 40 mm. In some embodiments, the diameter may be less than 30 mm. In some embodiments, the diameter may be less than 20 mm. In some embodiments, the diameter may be less than 15 mm. In some embodiments, the diameter may be less than 10 mm. In some embodiments, the diameter may be less than 5 mm. In a particular embodiment, the diameter is 14 mm.

In some embodiments, the contour may be in an oval shape. The diameter along a short axis of the oval may be 14 mm and the diameter along a long axis of the oval may be 32 mm.

In some embodiments, each segment or bridging member or boundary member may have a dimension of 0.1 by 0.1 mm. In some embodiments, the dimension may be 0.1 by 0.2 mm. In some embodiments, each segment, bridging member, or boundary member may have a dimension of equal to or less than 1 mm×1 mm. In some embodiments, each segment, bridging member, or boundary member may have a dimension of equal to or less than 0.75 mm×0.75 mm. In some embodiments, each segment, bridging member, or boundary member may have a dimension of equal to or less than 0.5 mm×0.5 mm.

In some embodiments, each segment, bridging member, or boundary member may have a dimension of equal to or greater than 0.25 mm×0.25 mm. In some embodiments, each segment, bridging member, or boundary member may have a dimension of equal to or greater than 0.5 mm×0.5 mm. In some embodiments, each segment, bridging member, or boundary member may have a dimension of equal to or greater than 0.75 mm×0.75 mm.

In some embodiments, the segments of the lateral member or the lateral members may have a spacing of equal to or greater than 0.5 mm. In some embodiments, the segments of the lateral member or the lateral members may have a spacing of equal to or greater than 0.7 mm. In some embodiments, the segments of the lateral member or the lateral members may have a spacing of equal to or greater than 1.0 mm to prevent formation of continuous blist or bleb formation. In some embodiments, the spacing may be equal to or greater than 1.2 mm to prevent formation of continuous blist or bleb formation. In some embodiments, the spacing may be equal to or greater than 1.4 mm to prevent formation of continuous blist or bleb formation. In some embodiments, the spacing may be equal to or greater than 1.6 mm to prevent formation of continuous blist or bleb formation. In some embodiments, the spacing may be equal to or greater than 1.8 mm to prevent formation of continuous blist or bleb formation. In some embodiments, the spacing may be equal to or greater than 2.0 mm to prevent formation of continuous blist or bleb formation. In some embodiments, the segments of the lateral member or the lateral members may have a spacing of equal to or less than 2.0 mm. In some embodiments, the segments of the lateral member or the lateral members may have a spacing of equal to or less than 1.8 mm. In some embodiments, the segments of the lateral member or the lateral members may have a spacing of equal to or less than 1.6 mm. In some embodiments, the segments of the lateral member or the lateral members may have a spacing of equal to or less than 1.4 mm. In some embodiments, the segments of the lateral member or the lateral members may have a spacing of equal to or less than 1.2 mm. In some embodiments, the segments of the lateral member or the lateral members may have a spacing of equal to or less than 1.0 mm. In some embodiments, the segments of the lateral member or the lateral members may have a spacing of equal to or less than 0.8 mm. In some embodiments, the segments of the lateral member or the lateral members may have a spacing of equal to or less than 0.6 mm.

In various aspects, the implant can have any variation as described in U.S. Pat. Nos. 6,962,573 and 7,806,847, incorporated herein by reference in their entirety.

Device Elasticity and Shape Memory Materials

The implant can return to its original shape inside the eyeball once implanted, as described in the procedure above. The ability of the ophthalmic implant to return to its original shape is due to the selection of the material as a shape memory material.

In some variations, at least the distal portion of the ophthalmic implant may be formed of a shape memory material. Alternatively, the entirety of the ophthalmic implant can be a shape memory material is a material that can revert back to its original shape after being bent, twisted, stretched, or otherwise manipulated. In some variations, the ophthalmic device, whether formed in whole or in part of a shape memory material, returns to its original shape without an external stimulus.

At least the distal portion of the ophthalmic implant is formed of a shape memory material, though it will be recognized that the entirety of the ophthalmic implant can be made of one or more shape memory materials. The shape memory material can be flexible, stretchable, and/or otherwise deformable. Further, the shape memory material can be resistant to erosion of eye tissues when the implant is in contact with the eye. The material may change its shape under an external force and may restore its shape once the external force is withdrawn or removed. The material may vary in compliance. In various aspects, the shape memory materials can return to their original shape without any additional stimulus.

The shape memory material can be any shape memory material known in the art. In some aspects, the shape memory material can be a shape memory alloy. For example, the shape memory alloy can be nitinol or a derivative thereof, copper-zinc-aluminum-nickel shape memory alloys, or copper-aluminum nickel shape memory alloys (Johnson Matthey Medical Components).

The shape memory material can be a shape memory polymer. Any shape memory polymer materials known in the art can be used. In some variations, the shape memory polymer is rubber. In other embodiments, the shape memory polymer is silicone. In still other aspects, the shape memory polymer can include styrene copolymers, such as disclosed in U.S. Pat. No. 6,759,481, copolymers described in U.S. Pat. No. 6,986,855, or maleimide-based high-temperature polymers described in U.S. Pat. No. 7,276,195 (Cornerstone Research, Dayton Ohio).

In further variations, the shape memory material can be coated. Any coating known in the art can be used. One or more shape memory materials can be used.

The ophthalmic implant formed of shape memory material can be stretched, rolled, folded, and/or otherwise deformed, so that it can be placed in a more confined space, such as an insertion device. In some embodiments, the implant can be rolled or folded to reduce its size from an original flat shape. The rolled implant may be in a rod shape. In some instances, the rolled or folded implant can be less than 3 mm in diameter. In some instances, the rolled or folded implant can be less than or equal to 2 mm in diameter. In some instances, the rolled or folded implant can be less than or equal to 1 mm in diameter

Implant Insertion

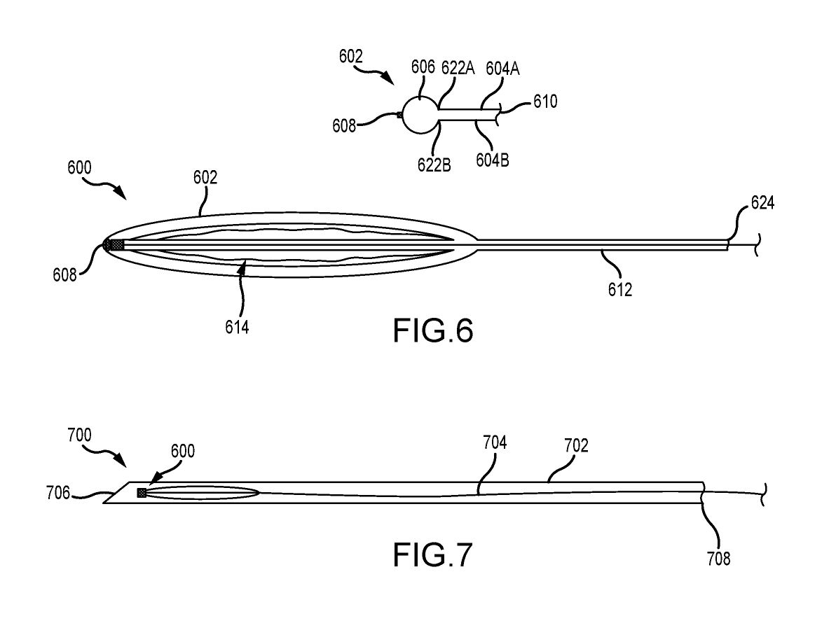

The implant can be inserted into the eyeball in various embodiments. In some embodiments, the implant can be stretched, rolled, folded, and/or otherwise deformed, to change its shape from an original flat structure to a longer shape with reduced width. For example, the stretched, rolled, folded, and/or otherwise deformed ophthalmic implant can be inserted into a catheter or a needle. FIG. 6 is a side view of an implant including a flat structure coupled to a fluid channel being stretched by wires in accordance with embodiments of the disclosure. As shown in FIG. 6, the flat structure can be stretched with a wire 602 to form an elongated implant structure 614. In some embodiments, the wire 602 includes a loop 606 with two opposite ends 622A-B coupled to two linear wire sections 604A-B, which may be are substantially parallel to each other. The loop 606 includes two sections 606A and 606B. The point 608 is at the middle point of the loop 606 to divide the loop 606 into two equal sections 606A and 606B. Alternatively, the ophthalmic implant can be rolled, folded, and/or otherwise deformed.

The un-deformed device, as shown in FIGS. 1-5 and 10, can be stretched by first placing the linear sections 604A-B of the wire 602 inside the fluid channel 612, followed by pulling the flat structure at a point 608 on the loop 606 and at an outer end 624 of the fluid channel 612 with the wire 602 together to form a stretched implant assembly 600 including an elongated implant structure 614 connected to the fluid channel 612, and the wire 612.

The wire 612 can help load the stretched implant assembly 600 into a needle. FIG. 7 is a side view of the implant of FIG. 6 inserted in a needle in accordance with embodiments of the disclosure. As shown in FIG. 7, the stretched implant assembly 600 is loaded or placed into a needle 702 to form a loaded implant assembly 700, which can be inserted into an eye tissue.

In some embodiments, the implant can be rolled, folded, or otherwise deformed to change its shape from an original flat shape to a rod-like shape and thus to reduce its size. The rolled or folded implant can be inserted into a catheter or needle. The loading component can be made of any other material that can be used to push the rod-like assembly into the lumen of the elongated member.

In order to have minimal or reduced invasiveness to the eye tissue, the catheter may have a small incision, much smaller than a common incision which is typically over 15 mm. In some embodiments, the incision of the catheter may be less than 3 mm. In some embodiments, the incision of the catheter may be less than 2 mm. In some embodiments, the incision of the catheter may be less than 1 mm.

The disclosure also provides an implant device for reduced, or minimally, invasive implantation. By minimally invasive, in various aspects, the implant device can be implanted with less injury to the body than would be required to implant a full size device. Minimally invasive implantation can result in implantation with less pain and scarring. It will be understood that “minimally” invasive does not mean the smallest injury possible, but rather less injury than in the full size device.

In some variations, the device is implanted via an elongated member having a distal end and a proximal end and a lumen between the distal end. In some embodiments, the elongated member may be a needle or a catheter. The distal portion 614 can be stretched from the proximal portion 612 by using the wire 602 such that the distal portion of the implant is deformed into a rod shape from an original shape (e.g. flat structure), which can be easily loaded into a needle. The wire 602 can be attached to the flat structure and extend to the end of the proximal portion or fluid channel 612.

The needle 702 includes a central lumen or a hollow central portion 704. In some embodiments, the lumen 704 may be in a cylindrical shape. The distal end 706 of the needle 702 is angled such that the distal end 706 can easily invade into a tissue, such as a human eye. The proximal end 708 is opposite to the distal end 706. The needle 702 may be formed of metal resistant to erosion in the tissue, such as stainless steel.

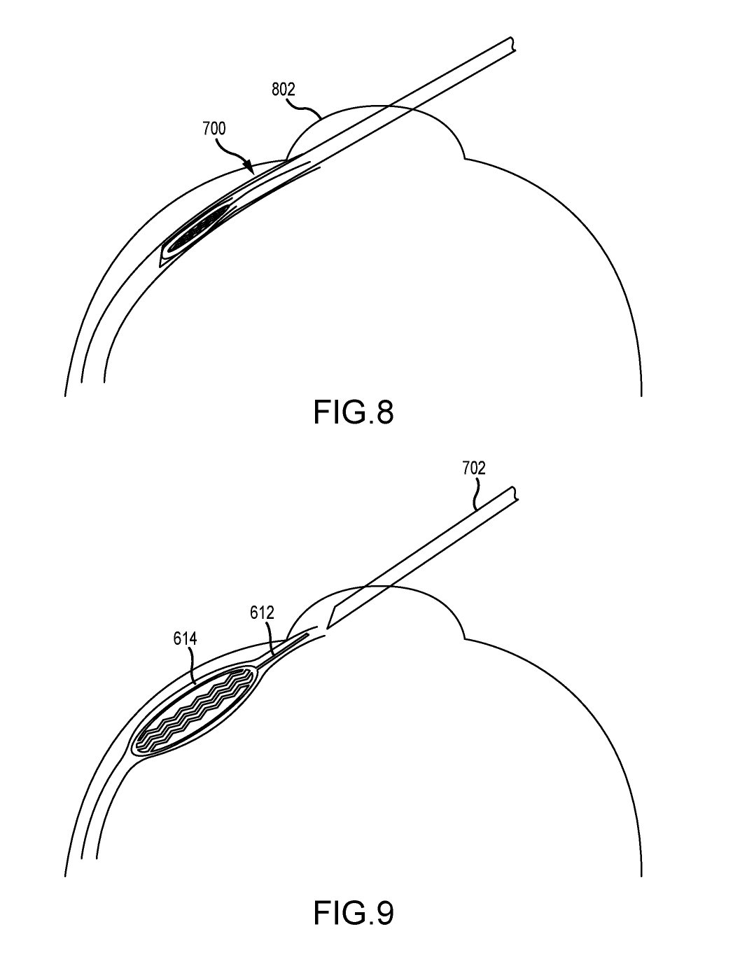

FIG. 8 illustrates that a loaded implant assembly 700 including the elongated implant structure, as shown in FIG. 7, inserted into eye tissue or eyeball 802 in accordance with embodiments of the disclosure. As shown in FIG. 8, the loaded implant assembly 700 including the needle 702 may be injected into an eye tissue 802 to place the elongated implant structure 614 and fluid channel 612.

When the loaded implant assembly 700 is inserted into the eye tissue 802, the needle 702 can be withdrawn to leave the elongated implant structure 614 and fluid channel 612 in place. FIG. 9 illustrates the needle 702 of FIG. 7 being withdrawn from the eye tissue 802 in accordance with embodiments of the disclosure. After the needle 702 is withdrawn, the wire 602 can be pulled out from the eye tissue 802 and the elongated implant structure 614 can recover its original shape, e.g. the flat structure. The flat structure provides large surface areas inside the tissue to help reduce intraocular pressure. The elongated implant structure 614 is placed in the eye tissue with the fluid channel 612 facing toward the eyeball 802.

When the end 624 of the fluid channel 612 is released, for example, by a plug, aqueous humor can flow from the fluid channel to the distal portion. The aqueous humor can flow through the fluid channel 612 toward the elongated implant structure 614. The aqueous humor inflates the filtration capsule, which can filter the aqueous humor from the fluid channel to reduce intraocular pressure.

In another embodiment, one or more dissolvable/replaceable plugs with optionally different dissolution profiles may be placed in the fluid channel to control pressure reduction.

In a further embodiment, one or more dissolvable/replaceable plugs with optionally different dissolution profiles may be placed in the proximal portion to control pressure reduction.

In an alternative embodiment, a plug may be placed before or after implantation in the eye to control pressure reduction. The plug may include a clotting agent or a hydrogel.

Referring to FIG. 11 again, the implant may be loaded into the needle 1120 by using the straight wire 1118. The wire 1118 can pass through the fluid channel 1106 and stop at a distal end 1114. The wire 1118 can help push the implant 1100 into the needle 1120. The needle 1120 may include an angled distal end 1128 for assisting inserting the needle into the eye tissue. The lumen 1124 is between the distal end 1128 and a proximal end 1126. In some embodiments, the lumen 1124 may be in a cylindrical shape.

The implant is loaded into the needle 1120 by first entering the distal portion of the implant 1100 into the lumen and then entering the fluid channel 1106. The loaded implant is deformed from its original shape into a rod-like shape to fit inside the lumen.

The rolled, folded, or stretched implant that is formed of a shape memory material changes from its original flat shape to a rod-like shape. By rolling, folding, or stretching, the implant can have a reduced size, such that the rolled, folded, or stretched implant can fit inside the lumen of the elongated member such as a needle or a catheter. The implant can return to its original shape, e.g. flat structure once the needle and the wire are removed.

It will be appreciated by those skilled in the art that the implant as illustrated in FIGS. 1-5 and 10 can also be pushed into a needle by using the straight wire 1118. The implant may be modified by adding a stop end.

It will be appreciated by those skilled in the art that any other implants may be stretched and loaded into the needle and thus can be implanted in the eyes with minimal invasiveness. For example, U.S. Pat. No. 7,806,847, entitled “C-Shaped Cross Section Tubular Ophthalmic Implant for Reduction of Intraocular Pressure in Glaucomatous Eyes and Method of Use,” by Michael Wilcox, discloses an implant, which is incorporated by reference in its entirety. The implant disclosed in U.S. Pat. No. 7,806,847 can also be loaded into the needle of the disclosure.

Any other existing competitor devices can be loaded into the needle. The devices described herein can also be used to design the device in the same shape and style as other conventional devices. The devices described herein can be adapted to have the same size and shape as the below conventional devices. The devices herein have the additional properties of creating reduced pressure on the eyeball as described herein, as well as being adaptable to minimally invasive administration, controlling diffusion and/or the rate thereof, introduction of clotting agents.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. An ophthalmic implant for reducing intraocular pressure in an eye tissue, the implant comprising:
a proximal portion comprising a fluid channel;
a distal portion comprising a filtration capsule forming member;
the filtration capsule forming member comprising at least one flat surface, and configured to form a cylindrical filtration capsule from the eye tissue surrounding the filtration capsule forming member when implanted;
wherein the fluid channel is configured to be fluidly connected to the cylindrical filtration capsule.

2. The ophthalmic implant of claim 1, wherein the filtration capsule forming member comprises a plurality of lateral members, a boundary member, a plurality of bridging members, or a combination thereof.

3. The ophthalmic implant of claim 2, wherein each of a plurality of lateral members, a boundary member, and a plurality of bridging members comprises a cross-section having a square or rectangular shape.

4. The ophthalmic implant of claim 1, wherein the distal portion comprises a plurality of lateral members within a circular or an elliptical contour.

5. The ophthalmic implant of claim 1, wherein the filtration capsule forming member comprises the plurality of lateral members.

6. The ophthalmic implant of claim 1, wherein the distal portion comprises a boundary member, a longitudinal bridging member, and a plurality of transverse bridging members connected between the boundary member and the longitudinal bridging member.

7. The ophthalmic implant of claim 1, wherein the filtration capsule forming member comprises the boundary member, the longitudinal bridging member, and the plurality of transverse bridging members.

8. An implantation device for implanting an ophthalmic implant into an eye tissue, the device comprising:
an elongated member comprising a distal portion and a proximal portion, and a lumen between the distal portion and the proximal portion, the ophthalmic implant according to claim 1 is placed in the lumen,
wherein the distal portion of the ophthalmic implant is in the distal portion of the needle, and the proximal portion of the ophthalmic implant is in the proximal portion of the needle.

9. The implantation device of claim 8, wherein the elongated member comprises a needle.

10. The implantation device of claim 8, further comprising a loading component configured to mechanically push the ophthalmic implant into the lumen.

11. The implantation device of claim 10, wherein the loading component is configured to guide the ophthalmic implant toward the distal portion of the elongated member.

12. The implantation device of claim 10, wherein the loading component comprises a metal wire configured to pass through the fluid channel of the ophthalmic implant.

13. The implantation device of claim 12, wherein the ophthalmic implant comprises a stop end configured for the wire to be pushed against to allow the ophthalmic implant to be pushed into the lumen.

14. The implantation device of claim 8, wherein the loading component comprises a metal wire configured to stretch the ophthalmic implant to shape the ophthalmic implant into a rod-like assembly comprising the implant and wire, such that the rod-like assembly can be pushed into the lumen of the elongated member.

15. The implantation device of claim 8, wherein the ophthalmic implant comprises a shape memory material.

16. A method for implanting the ophthalmic implant, the method comprising:
inserting an elongated member loaded with the ophthalmic implant of claim 1 into an eye ball, wherein the ophthalmic implant is deformed from an original shape to fit inside the elongated member; and
withdrawing the elongated member from the eye ball to leave the ophthalmic implant in the eye ball, wherein the ophthalmic implant returns to the original shape.

17. The method of claim 16, the method further comprising loading the ophthalmic implant into a lumen of an elongated member by pushing a loading component, wherein the ophthalmic implant is deformed from the original shape to fit inside the lumen.

18. The method of claim 17, the method further comprising pulling out the loading component after withdrawing the elongated member.

19. An ophthalmic implant for reducing intraocular pressure in an eye tissue, the implant comprising:
a proximal portion comprising a fluid channel; and
a distal portion comprising a filtration capsule forming member, the filtration capsule forming member configured to form a cylindrical filtration capsule when the ophthalmic implant implanted,
wherein the distal portion comprises a shape memory material; and
wherein the fluid channel is configured to be fluidly connected to the cylindrical filtration capsule when formed from the eye tissue surrounding the filtration capsule forming member.

20. The ophthalmic implant of claim 19, wherein the filtration capsule forming member has at least on one flat surface.
